(19) United States
(12) Patent Application Publication
(10) Pub. No.: US 2006/0034874 A1
(43) Pub. Date: Feb. 16, 2006

(54) Composition for lessening malodors during hair treatment and hair removal and method of use

(76) Inventors: Anthony E. Winston, East Brunswick, NJ (US); Carl Mayer, Woodbury Heights, NJ (US)

Correspondence Address: CHURCH & DWIGHT CO., INC., LAW DEPT. - PATENTS, 469 NORTH HARISON STREET, PRINCETON, NJ, 08543-5297, US

(21) Appl. No.: 10/520,650

(22) PCT Filed: Jul. 7, 2003
(86) PCT No.: PCT/US03/20994

§ 371 Date: Jan. 7, 2005

Related U.S. Application Data
(60) Provisional application No. 60/394,770, filed on Jul. 10, 2002.

(51) Int. Cl. A61K 8/46; A61K 8/02

(52) U.S. Cl. 424/401

(57) ABSTRACT

The use of strontium ions to reduce malodors associated with various chemical hair removal and hair treatment procedures is disclosed. Typical formulations include alkali metal thioglycolate depilatories containing from 0.2% to 2% strontium chloride.

### CROSS REFERENCE TO RELATED APPLICATION

This application claims priority based on a U.S. Provisional Patent Application filed Jul. 2, 2002, titled “Composition For Lessening, Eliminating or Preventing Malodors and Method of Use”.

### FIELD OF THE INVENTION

This invention is directed to the use of strontium ions to lessen or eliminate malodors due to sulfides during certain hair treatment and hair removal procedures. Such malodors may be formed, for example, when S—S bonds are broken in connection with hair depilation. The invention is, however, useful in connection with the minimization of sulfide odors arising from other sources and processes.

### BACKGROUND OF THE INVENTION

The removal of hair from the human body has received considerable attention. The hair and hair follicles can be removed by certain surgical operations or by electrolysis. Also, it is customary to remove hair by the use of tweezers or other instruments but does not have the same long lasting effects as surgical procedures. Moreover, the use of hair removal instruments is generally confined to removal of hair from a localized area, such as, along the eyebrows or nostrils.

Creams or cold waxes have been formulated in the past for the purpose of hair removal to the end of achieving more lasting effects as well as the ability to apply over greater areas than is possible by plucking out individual hairs from a localized part of the body. For example, it has been proposed in the past to employ a combination of honey, rosin and wax which are heated together and thereafter combined with citric acid, which is mixed into the composition until it has acquired a creamy texture, reference being made to U.S. Pat. No. 2,091,313 to W. M. Grant Grant fails to state to what temperature level the formulation is heated.

British Letters Patent No. 901,624 to E. Wenden discloses the formulation of a cream made up of sugar and lemon juice, glycerine, boric acid powder, sodium chloride and a water carrier. These ingredients are heated, then allowed to cool to a temperature at which they may be poured into separate jars or containers, and specifically are heated to a temperature on the order of 278° F. to form a plastic mass. The resultant composition is applied to the skin so as to become matted with the hair, then immediately stripped from the skin to cause removal of the hair with the plastic mass.

Other methods of hair removal have employed naturally occurring chemical substances, such as certain plant extracts, which have the ability to cause the removal of human body hair when applied to a particular skin surface. Some of these naturally occurring chemical depilatories may be are quite harsh, causing substantial irritation to human skin, particularly to sensitive skin areas such as the face, and are frequently malodorous as well.

In conjunction with waxes of various sorts, the most widelyused present day depilatory agents are probably creams and ointments which contain as their active ingredient disulfide bond breakers such as salts of mercaptan acids, particularly alkali metal and alkali earth metal salts of thioglycolic acid. These sales attack the most recently formed portion of the hair shaft, that is, the part closest to the skin surface. They act by rupturing the disulfide bonds in the keratin protein found in the hair shaft, weakening the hair and causing it to be ruptured at the surface of the skin, an effect somewhat similar to closely shaving the skin area. The hair root, however, remains in the follicle and the disulfide bond breaking salts have a deleterious effect on the skin and give rise to unpleasant odors. One aspect of the present invention involves the reduction of these unpleasant odors.

### SUMMARY OF THE INVENTION

1. Objects of the Invention.

It is an object of the present invention to provide compositions for use in the removal of human hair that are safe, effective, non-irritating and non-toxic and that can be utilized in simple and inexpensive hair-removal methods. It is a further object of the invention to chemically remove human hair in a manner with less associated malodors than has heretofore been accomplished. There and other objects of the invention will become apparent from the description set forth herein.

2. Brief Description of the Invention.

Deodorization of Depilatories and Hair Bleaches.

This invention provides for a means of deodorizing hair treatment and hair removal processes. More particularly this invention covers means of preventing formulations containing compounds with mercaptan moieties from becoming odiferous during the course of hair treatment and hair removal. More particularly this invention covers methods of deodorizing depilatories, hair permanents and hair straighteners. It also covers the prevention of odor development when such products are applied to hair and skin and areas being depilated.

As described above, many commercial depilatories utilize alkali metal and alkali earth metal thioglycolates as reducing agents to weaken hair for the purposes of depilation. When formulated into depilatory products, thioglycolates tend to decompose over time releasing hydrogen sulfide gas, which has an unpleasant odor similar to “bad eggs”. Additionally, the reactions of depilatories with hair produce hydrogen sulfide and methyl mercaptan, thereby creating malodors during depilation. Similarly products designed to permanently or temporarily weaken hair such as permanents and products react with the sulfur-containing amino acids in hair and generate odiferous sulfides.

Copper and lead are known to react with sulfides producing insoluble non-volatile compounds. However, both copper and lead are toxic and produce dark colored products when reacted with sulfides, making them unsuitable for use in personal care products. Copper and possibly lead also react with mercaptans making them potentially incompatible with thioglycolates. Silver blackens and precipitates as silver hydroxide from alkaline solutions. It also darkens the skin making it unsuitable for use in personal care products. Silver also strongly reacts with most sulfur-containing compounds, making it incompatible with thioglycolates.

The present invention is directed to formulations containing thioglycolates together with sources of strontium ions at concentrations, which reduce or eliminate the generation of sulfide odors from the formulation. It also is directed to depilatories, perms and hair straighteners containing sources of strontium ions to prevent the development of sulfur-containing malodors when the products are contacted with hair.

Depilatory formulations containing thioglycolates generate sulfide odors on aging. We have now found that when a source of strontium is added to such depilatories, the development of sulfide odors on aging is significantly reduced or eliminated. Additionally, when applied to the skin for the purposes of removing hair, sulfide odors are not generated and the skin remains odor-free. Furthermore, formulations containing sources of strontium ions were not found to adversely affect the efficacy of the depilatory formulation. While not being bound by any particular theory of operation, we hypothesize that strontium ions react with any free sulfide ion to produce insoluble strontium sulfide and thereby prevents the formation of hydrogen sulfide and hence the release of malodor.

Any source of strontium ions is effective for incorporation in the formulation. However, soluble non-toxic compounds such as strontium acetates and chlorides are preferred. The level of strontium compound used should be sufficient to provide enough strontium ions to prevent the development of sulfide odor, for example from about 0.05 to 8%, more preferable from 0.1 to 4%, most preferably 0.2 to 2%, wherein all percentages are by weight of the respective metal ions.

The following is an example of a hair depilatory agent prepared according to the instant invention.

### EXAMPLES

| | Example 1 | Control A | Control B |
|---|---|---|---|
| Water | 64.0 | 64.0 | 64.0 |
| Potassium thioglycolate | 11.5 | 11.5 | 11.5 |
| Calcium hydroxide | 5.0 | 5.0 | 5.0 |
| Cetearyl alcohol | 4.5 | 4.5 | 4.5 |
| Light mineral oil | 8.0 | 8.0 | 8.0 |
| Sodium hydroxide | 1.5 | 1.5 | 1.5 |
| Urea | 5.0 | 5.0 | 5.0 |
| Strontium chloride | 0.5 | 0.0 | 0.0 |
| Zinc oxide | 0.0 | 0.5 | 0.0 |
| | 100.0 | 100.0 | 99.5 |

The products stored for two months at room temperature and the odor of the product in the container was evaluated. Example 1 and Control A were essentially odor-free. The Control B had quite a perceptible unpleasant odor.

The three products were then applied to areas of the skin for depilation. Example 1 and Control B were both effective in removing the hair. Control B was significantly less effective in removing hair. On the other hand Example 1 and Control A did not produce an odor whereas Control B did. Also the treated skin after use of Example 1 and Control A was odor free whereas the skin treated with Control B had an unpleasant odor.

This demonstrates that strontium ions effectively prevent odor development from the depilatory formulations without adversely affecting depilation performance. In contrast, while zinc ions do prevent odor, they also interfere with depilation performance.

### FURTHER EXAMPLES

A hair removal product for men having the below listed composition, stability and viscosity was prepared according the following procedure.

| Ingredient | Wt % |
|---|---|
| Dionized water | 73.475 |
| Calcium Thioglycolate | 6.000 |
| Calcium Hydroxide, powder | 2.500 |
| Sodium Hydroxide, bead | 1.000 |
| Cetearyl Alcohol and Ceteareth-20 | 8.000 |
| (a.k.a. Promulgen D) | |
| Light Mineral Oil | 7.000 |
| Chromium Hydroxide Green | 0.025 |
| Perfume | 0.500 |
| Sunflower Oil | 0.500 |
| Camelia Oleifera Extract | 0.500 |
| Strontium Chloride | 0.500 |
| Total | 100.000 |

Procedure:

1. While stirring heat de-ionized water and add calcium thioglycolate.

2. When the temperature reaches about 55 °C., add calcium hydroxide.

3. When the temperature of the mixture reaches about 65 °C., slowly add the sodium hydroxide.

4. When the temperature reaches 75 °C., add the Promulgen D. Then add Light Mineral Oil, Chromium Hydroxide Green, Sunflower Oil, and Camelia Extract and stir for a further 5 minutes)

5. Remove heat while continuing to stir.

A similar control product was also prepared without strontium chloride. A sample of the product containing strontium chloride is placed on stability test with the control product, not containing strontium chloride. After 3 months at 100° F. the test product has a pleasant fragrance, while the control product, not containing strontium chloride, has an unpleasant off-odor similar to bad eggs.

Fresh product containing strontium chloride is applied to the legs, chest and back of male subjects. It is allowed to remain in contact with the hair for 8 minutes. The product is then wiped off the skin and the hair is effectively removed. The skin remains essentially odor free. The control product not containing strontium chloride is also found to be effective in removing hair. However, an unpleasant lingering odor is imparted to the skin.

A hair removal product for women having the below listed composition, stability and viscosity was prepared according to the following procedure.

| Ingredient | Wt % |
|---|---|
| Dionized water | 63.435 |
| Potassium Thioglycolate | 11.500 |
| Calcium Hydroxide | 5.000 |
| Urea | 5.000 |
| Sodium Hydroxide | 1.500 |
| Cetearyl Alcohol and Ceteareth-20 | 4.500 |
| (a.k.a. Promulgen D) | |
| Light Mineral Oil | 8.000 |
| D&C Yellow #8 | 0.005 |
| Chromium Hydroxide Green | 0.060 |
| Perfume Compound CH15736 | 0.500 |
| Strontium Chloride | 0.500 |
| Total | 100.000 |

Procedure:

1. While stirring heat de-ionized water and add potassium thioglycolate.

2. When the temperature reaches approximately 55 °C., add calcium hydroxide.

3. When the temperature of the batch reaches 65 °C., slowly add the urea followed by the sodium hydroxide.

4. When the temperature reaches 75 °C., add the Promulgen D followed by the light mineral oil, yellow dye and chromium hydroxide green and stir for 5 minutes.

5. Remove heat and allow to cool.

6. When the temperature drops to 56 °C., add the perfume and stir for 1 minute.

7. Homogenize the batch for 1 minute.

8. Mix and cool to ˜40 °C.

9. Add the strontium chloride and mix for a further 5 minutes until contents cool to 30 °C.

A similar control product not containing strontium chloride was perpared using the same procedure.

A sample of the Example product containing strontium chloride is placed on stability test with the control product, not containing strontium chloride. After 3 months at 100° F. the test product has a pleasant fragrance, while the control product, not containing strontium chloride, has an unpleasant off-odor similar to bad eggs.

Fresh product is applied to the legs of female subjects and left in contact with the hair for 4 minutes. The product is wiped off the skin. The hair iseffectively removed while leaving the skin essentially odor-free. When a control product, not containing strontium chloride, is applied to the legs it is also effective in removing the hair. However, there is a perceptible unpleasant odor which lingers on the skin.

We claim:

1. A chemical depilatory composition comprising at least one chemical depilatory component and an effective sulfide malodor lessening, eliminating or preventing amount of strontium ions.

2. The composition of claim 1 wherein the chemical depilatory is selected from the group consisting of alkali or alkali earth thioglycolates.

3. The composition of claim 1 wherein the said strontium ions are present in the form of a soluble, non-toxic strontium salt in an amount ranging from about 0.2% by weight of said salt up to about 2% by weight of said salt.

4. The composition of claim 3 wherein said chemical depilatory component is selected from the group comprising potassium thioglycolate, calcium thioglycolate and mixtures thereof and said strontium compound is strontium chloride.

5. A method for removing hair from the surface of human skin comprising applying to the surface of said skin the composition of claim 1.
